(19) United States
(12) Patent Application Publication
(10) Pub. No.: US 2002/0004043 A1
(43) Pub. Date: Jan. 10, 2002

(54) Cellular and animal models for diseases associated with altered mitochondrial function

(76) Inventors: Christen M. Anderson, Encinitas, CA (US); William Clevenger, Vista, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/887,765

(22) Filed: Jun. 21, 2001

Related U.S. Application Data
(63) Continuation of application No. 09/069,489, filed on Apr. 28, 1998.

(51) Int. Cl. A61K 48/00; A01N 63/00

(52) U.S. Cl. 424/93.21; 435/455; 800/21

(57) ABSTRACT

The present invention provides methods for depleting mitochondrial DNA from insulin secreting cells using antiviral compounds, and for producing mitochondrial cytoplasmic hybrid (“cybrid”) cells and animals from mitochondrial DNA depleted cells. Also provided are methods for modeling diseases associated with altered mitochondrial function, including NIDDM, and methods for diagnosis of such diseases and methods for screening agents that may be useful for such diseases.

### TECHNICAL FIELD

The present invention relates generally to model systems for diseases that involve defects in the function of mitochondria, where those defects arise from defects in the genes that regulate mitochondrial structure and activity.

### BACKGROUND OF THE INVENTION

A number of degenerative diseases are thought to be caused by or to be associated with alterations in mitochondrial metabolism. These include diabetes mellitus, Alzheimer's Disease, Parkinson's Disease, Huntington's disease, dystonia, Leber's hereditary optic neuropathy (LHON), schizophrenia, and myodegenerative disorders such as “mitochondrial encephalopathy, lactic acidosis, and stroke” (MELAS), and “myoclonic epilepsy ragged red fiber syndrome” (MERRF).

Type II diabetes mellitus is a common degenerative disease affecting 5 to 10 percent of the population in developed countries. It is a heterogenous disorder with a strong genetic component; monozygotic twins are highly concordant and there is a high incidence of the disease among first degree relatives of affected individuals. The propensity for developing type II diabetes mellitus is reportedly maternally inherited, suggesting a mitochondrial genetic involvement. (Alcolado, J. C. and Alcolado, R., Br. Med. J. 302:1178-1180 (1991); Reny, S. L., International J. Epidem. 23:886-890 (1994)).

Studies have shown that diabetes mellitus may be preceded by or associated with certain related disorders. For example, it is estimated that forty million individuals in the U.S. suffer from late onset impaired glucose tolerance (IGT). Individuals with IGT fail to secrete insulin normally in response to a glucose challenge. A small percentage of IGT individuals (5-10%) progress to non-insulin dependent diabetes (NIDDM) each year. Some of these individuals eventually require therapy with insulin. This form of diabetes is associated with impaired release of insulin by pancreatic beta cells and/or a decreased end-organ response to insulin. Complications of diabetes mellitus and conditions that precede or are associated with diabetes mellitus include: obesity, vascular pathologies, peripheral and sensory neuropathies, blindness, and deafness.

Due to the strong genetic component of diabetes mellitus, the nuclear genome has been the main focus of the search for causative genetic mutations. However, despite intense effort, nuclear genes that segregate with diabetes mellitus are rare and include, for example, mutations in the insulin gene, the insulin receptor gene, the adenosine deaminase gene and the glucokinase gene. Altered mitochondrial genes that segregate with diabetes mellitus are disclosed generally in PCT/US95/04063.

A growing body of evidence suggests that the genetic basis of NIDDM resides in mitochondrial DNA rather than in the nucleus. For example, NIDDM exhibits a predominantly maternal pattern of inheritance and is also present in diseases known to be based on a mitochondrial DNA (mtDNA) defect. Approximately 1.5% of all diabetic individuals, for instance, harbor a mutation at mtDNA position 3243 in the mitochondrial gene encoding leucyl-tRNA (tRNA^{Leu}). This mutation is known as the MELAS (mitochondrial encephalopathy, lactic acidosis and stroke) mutation. (Gerbitz et al., Biochim. Biophys. Acta 1271:253-260, 1995.) Similar theories have been advanced for analogous relationships between mtDNA mutations and other neurological diseases, including but not limited to Leber's hereditary optic neuropathy (LHON), schizophrenia, and myoclonic epilepsy ragged red fiber syndrome (MERRF). It is plausible that other mtDNA mutations are associated with the common form of NIDDM. Identification of such mutations and their functional consequences may provide targets for development of therapeutic agents.

Functional mitochondria contain gene products encoded by mitochondrial genes situated in mtDNA and by extramitochondrial genes such as those found in nuclear DNA. Accordingly, mitochondrial and extramitochondrial genes may interact directly, or indirectly via gene products and their downstream intermediates including but not limited to metabolites, catabolites, substrates, precursors, cofactors and the like. Alterations in mitochondrial function, for example impaired electron transport activity, defective oxidative phosphorylation or increased free radical production, may therefore arise as the result of defective mtDNA, defective extramitochondrial DNA, defective mitochondrial or extramitochondrial gene products, defective downstream intermediates or a combination of these and other factors.

Regardless of whether a defect underlying altered mitochondrial function may have mitochondrial or extramitochondrial origins, and regardless of whether a defect underlying altered mitochondrial function has been identified, the present invention provides methods that are useful for modeling diseases associated with such altered mitochondrial function.

The identification of therapeutic regimens or drugs that are useful in the treatment of disorders associated with altered or defective mitochondrial function such as those described above has historically been hampered by the lack of reliable model systems that could be used for rapid and informative screening of candidate compositions. Animal models do not exist for many of the human diseases that are associated with altered or defective mitochondrial function or mitochondrial gene defects. In addition, appropriate cell culture model systems are either not available, or are very difficult to establish and maintain. Furthermore, even when cell culture models are available, it is often not possible to discern whether the mitochondrial or the cellular genome is responsible for a given phenotype, because mitochondrial functions may often be encoded by both genomic and mitochondrial genes as described above. It is therefore also not possible to tell whether the apparent effect of a given drug or treatment operates at the level of the mitochondrial genome or elsewhere.

In order to determine whether a mitochondrial gene defect may contribute to a particular disease state, it may be useful to construct a model system in which the nuclear genetic background may be held as a constant while the mitochondrial genome is modified. It is known in the art to deplete mitochondrial DNA from cultured cells to produce p⁰ cells, thereby preventing expression and replication of mitochondrial genes and inactivating mitochondrial function. See, for example, International Publication Number WO 95/26973, which is hereby incorporated by reference in its entirety, and references cited therein. It is further known in the art to repopulate such p⁰ cells with mitochondria derived from foreign cells in order to assess the contribution of the donor mitochondrial genotype to the respiratory phenotype of the recipient cells. Such cytoplasmic hybrid cells, containing genomic and mitochondrial DNAs of differing biological origins, are known as cybrids. Additionally, for the production of cybrid cell lines it is known to generate p⁰ cells from undifferentiated, immortalized cell lines that can be induced to differentiate in vitro. Generation of cybrid animals by production of p⁰ embryonal cells that may be reintroduced into a surrogate mother for completion of gestation, is also known in the art.

Mitochondrial transformations of p⁰ cells to produce cybrids known in the art may not always have been done using cells of the types that are most affected by the particular mitochondria associated disease under investigation, making it unclear whether the mitochondrial deficiencies observed in the cybrid cells are related to the disease state being studied.

Clearly, there is a need for reliable model biological systems that may be useful for screening candidate therapeutic compositions and identifying those that may be suitable for treatment of mitochondria associated diseases, including but not limited to diabetes mellitus and neurodegenerative disorders. Such model systems may include in vitro models for these mitochondria associated diseases (e.g., a NIDDM cell line that exhibits impaired insulin secretion or decreased insulin responsiveness); they may also include animal models of these disorders (e.g., an animal model of diabetes mellitus). Reliable diagnoses of mitochondria associated diseases at their earliest stages are critical for efficient and effective intercession and treatment of these disorders, given their often debilitating nature. Accordingly, there is also a need for a non-invasive diagnostic assay that is reliable at or before the earliest manifestations of symptoms for any of the mitochondria associated diseases.

The present invention satisfies these needs for in vitro and in vivo model biological systems that are useful for the development of drug screening assays, diagnostic assays and effective treatment of mitochondria associated diseases, and provides related advantages as well.

### SUMMARY OF THE INVENTION

According to the present invention, model systems for diseases that involve altered mitochondrial function are provided. In one aspect, the invention provides a method of generating a p⁰ cell by contacting an insulin secreting cell with an antiviral compound. In another aspect, the invention provides a method of generating a mitochondrial DNA depleted cell by contacting an insulin secreting cell with an antiviral compound. In certain embodiments of these aspects of the invention, the antiviral compound is a nucleoside analog, which may in some further embodiments be 2′3′-dideoxycytidine, 3′-azido-3′deoxythymidine, dideoxyadenosine, dideoxyguanosine, dideoxythymidine, 2′3′-dideoxyinosine, 2′3′-didehydro-3′deoxythymidine, dideoxydidehydrothymidine, dideoxydidehydrocytidine, ganciclovir or acycloguanosine.

In some embodiments of the invention, the insulin secreting cell is an immortalized cell line, and in some embodiments the insulin secreting cell is capable of being induced to differentiate and/or is undifferentiated.

One aspect of the invention provides a method of producing a cybrid cell line, comprising the steps of treating an insulin secreting cell line with an antiviral compound to convert the cell line into a p₀ cell line, and then repopulating such a p⁰ cell line with isolated mitochondria to form a cybrid cell line. In one embodiment the cybrid cell line has extramitochondrial genomic DNA and mitochondrial DNA of differing biological origins. In a further embodiment the cybrid cell line has extramitochondrial genomic DNA and mitochondrial DNA from xenogeneic species. In a further embodiment the cybrid cell line has mitochondrial DNA from a rodent species, which may in further embodiments be mitochondrial DNA derived a mouse, rat, rabbit, hamster, guinea pig or gerbil. In one such further embodiment the cybrid cell line has mitochondrial DNA from a BHE/cdb rat.

It is another aspect of the invention to provide a method of producing a cybrid cell line, by treating an insulin secreting cell line with an antiviral compound to convert the cell line into a mitochondrial DNA depleted cell line, and then repopulating such a mitochondrial DNA depleted cell line with isolated mitochondria, to form the cybrid cell line. In certain embodiments of this aspect of the invention, the cybrid cell line has extramitochondrial genomic DNA and mitochondrial DNA of differing biological origins. In certain embodiments the cybrid cell line has extramitochondrial genomic DNA and mitochondrial DNA from xenogeneic species. In certain embodiments the cybrid cell line has mitochondrial DNA from a rodent species, which in certain further embodiments may be mitochondrial DNA from a mouse, rat, rabbit, hamster, guinea pig or gerbil. In one further embodiment the cybrid cell line has mitochondrial DNA from a BHE/cdb rat.

In certain embodiments of the invention, a cybrid cell line is produced by treating an insulin secreting cell line with an antiviral compound that is a nucleoside analog. In some embodiments the antiviral compound may be 2′3′-dideoxycytidine, 3′azido-3′deoxythymidine, dideoxyadenosine, dideoxyguanosine, dideoxythymidine, 2′3′-dideoxyinosine, 2′3′-didehydro-3′-deoxythymidine, dideoxydidehydrothymidine, dideoxydidehydrocytidine, ganciclovir or acycloguanosine.

In some embodiments of the invention, the insulin secreting cell line to be treated with an antiviral compound is an immortalized cell line. In certain embodiments the cybrid cell line produced according to the method provided is capable of secreting insulin. In certain embodiments the cybrid cell line produced according to the method provided is capable of responding to insulin. In certain embodiments the the cell line is derived from a pancreatic beta cell. In certain embodiments the cell line is an undifferentiated cell line that is capable of being induced to differentiate.

Some embodiments of the invention provide a method of producing a cybrid cell line using isolated mitochondria that are obtained from a subject known to be afflicted with a disorder associated with a mitochondrial defect. In some embodiments of the invention that provide methods of producing a cybrid cell line having extramitochondrial genomic DNA and mitochondrial DNA of differing biological origins, the extramitochondrial genomic DNA has its origin in an immortal cell line, and the mitochondrial DNA has its origin in a human tissue sample. In certain of these embodiments the human tissue sample is further derived from a patient having a disease that is associated with a mitochondrial defect.

It is another aspect of the present invention to provide a method of constructing an immortal cybrid cell line, comprising the steps of: treating an immortal insulin secreting cell line with an antiviral compound to convert the cell line into an immortal p⁰ cell line, and repopulating the immortal p⁰ cell line with mitochondria isolated from tissue of a patient afflicted with diabetes mellitus, Alzheimer's Disease, Parkinson's Disease, Huntington's disease, dystonia, Leber's hereditary optic neuropathy, schizophrenia, myoclonic-epilepsy-lactic-acidosis-and-stroke (MELAS), or myoclonic-epilepsy-ragged-red-fiber—syndrome (MERRF), to form the cybrid cell line.

It is another aspect of the invention to provide a method of constructing an immortal cybrid cell line by treating an immortal insulin secreting cell line with an antiviral compound to convert the cell line into an immortal mitochondrial DNA depleted cell line; and repopulating such an immortal mitochondrial DNA depleted cell line with mitochondria isolated from tissue of a patient afflicted with diabetes mellitus, Alzheimer's Disease, Parkinson's Disease, Huntington's disease, dystonia, Leber's hereditary optic neuropathy, schizophrenia, myoclonic-epilepsy-lactic-acidosis-and-stroke (MELAS), or myoclonic-epilepsy-ragged-red-fiber syndrome (MERRF), to form the cybrid cell line.

In another aspect of the invention, a method is provided for preparing a cybrid animal, by treating embryonic cells isolated from a multicellular, non-human animal with an antiviral compound to convert the cells to a p⁰ state, and then repopulating these p⁰ embryonic cells with mitochondria isolated from another cell source, to produce a cybrid animal.

In another aspect of the invention, a method is provided for preparing a cybrid animal, by treating embryonic cells isolated from a multicellular, non-human animal with an antiviral compound to convert the cells to a mitochondrial DNA depleted state, and then repopulating these mitochondrial DNA depleted embryonic cells with mitochondria isolated from another cell source, to produce a cybrid animal.

In another aspect, the invention provides a method of detecting a disease associated with altered mitochondrial function by treating an insulin secreting cell line with an antiviral compound to convert the cell line into a mitochondrial DNA depleted cell line or a p⁰ cell line, repopulating such a mitochondrial DNA depleted cell line or p⁰ cell line with mitochondria from a donor subject suspected of having a disease associated with altered mitochondrial function to produce a cybrid cell line, determining altered levels of insulin secretion by such a cybrid cell line and therefrom identifying the mitochondria donor subject as having a disease associated with altered mitochondrial function.

In another aspect, the invention provides a method of detecting a disease associated with altered mitochondrial function comprising treating an insulin secreting cell line with an antiviral compound to convert the cell line into a mitochondrial DNA depleted cell line or a p⁰ cell line, repopulating such a mitochondrial DNA depleted cell line or p⁰ cell line with mitochondria from a donor subject suspected of having a disease associated with altered mitochondrial function to produce a cybrid cell line, comparing altered levels of insulin secretion by such a cybrid cell line to insulin secretion by an insulin secreting cell line having mitochondria from a subject with normal mitochondrial function and therefrom identifying the mitochondria donor subject as having a disease associated with altered mitochondrial function.

In another aspect, the invention provides a method of evaluating an antiviral compound for its effect on mitochondrial function, by treating an insulin secreting cell line with an antiviral compound to convert the insulin secreting cell line into a mitochondrial DNA depleted cell line or a p⁰ cell line, repopulating the mitochondrial DNA depleted cell line or p⁰ cell line with mitochondria to produce a cybrid cell line, and determining insulin secretion by the cybrid cell line in the presence or absence of an antiviral compound, therefrom identifying an effect of the antiviral compound on mitochondrial function. In certain embodiments, the mitochondria are from a subject suspected of having a disease associated with altered mitochondrial function. In certain embodiments, the cybrid cell line has extramitochondrial genomic DNA and mitochondrial DNA of differing biological origins. In certain embodiments the cybrid cell line has extramitochondrial genomic DNA and mitochondrial DNA from xenogeneic species. In some embodiments the cybrid cell line has mitochondrial DNA from a rodent species. In some embodiments the cybrid cell line has mitochondrial DNA from a mouse, rat, rabbit, hamster, guinea pig or gerbil. In certain embodiments the cybrid cell line has mitochondrial DNA from a BHE/cdb rat.

In another aspect, the invention provides a method of identifying an agent that at least partially restores insulin secretion to a cell exposed to an antiviral compound which inhibits insulin secretion, comprising treating an insulin secreting cell line with an antiviral compound to convert the cell line into a mitochondrial DNA depleted cell line or a p⁰ cell line, repopulating such a mitochondrial DNA depleted cell line or p⁰ cell line with mitochondria to produce a cybrid cell line, contacting such a cybrid cell line with a candidate agent capable of at least partially restoring insulin secretion to the cybrid cell line, detecting an increase in insulin secretion by the cybrid cell line and therefrom identifying an agent that partially restores insulin secretion.

In another aspect, the invention provides a method for selecting a therapeutic agent suitable for use in a subject having a disease associated with altered mitochondrial function, comprising treating an insulin secreting cell line with an antiviral compound to convert the cell line into a mitochondrial DNA depleted cell line or a p⁰ cell line, repopulating such a mitochondrial DNA depleted cell line or p⁰ cell line with mitochondria from a subject having a disease associated with altered mitochondrial function to produce a cybrid cell line, detecting the level of insulin secretion by such cybrid cell line, contacting the cybrid cell line with a candidate therapeutic agent, detecting the effect of the candidate therapeutic agent on insulin secretion by the cybrid cell line and therefrom determining the suitability of the therapeutic agent.

In another aspect, the invention provides a method for selecting a suitable therapeutic agent for use in a subject having a disease associated with impaired insulin secretion, comprising treating an insulin secreting cell line with an antiviral compound to convert the cell line into a mitochondrial DNA depleted cell line or a p⁰ cell line, repopulating such a mitochondrial DNA depleted cell line or p⁰ cell line with mitochondria from a subject having a disease associated with impaired insulin secretion to produce a cybrid cell line, detecting the level of insulin secretion by the cybrid cell line, contacting the cybrid cell line with a candidate therapeutic agent, detecting the effect of the candidate therapeutic agent on insulin secretion by the cybrid cell line and therefrom determining the suitability of the therapeutic agent.

The model systems described herein offer outstanding opportunities to identify, probe and characterize defective mitochondrial genes and mutations thereof, to determine their cellular and metabolic phenotypes, and to assess the effects of various drugs and treatment regimens in vitro and in vivo. Because such cell-based model systems are observed to undergo phenotypic changes characteristic of the diseases to which they relate, they can also be used in methods of diagnosis. By using these same cell cultures and/or animal models according to the invention in screening assays, it is also possible to predict which of several possible drugs or therapies may be desirable for a particular patient.

These and other aspects of the invention will become more apparent by reference to the following detailed description of the invention and attached drawings.

### BRIEF DESCRIPTION OF THE DRAWINGS

FIG. 1 illustrates the effect of exposure to various concentrations of three representative antiviral compounds for seven days on the relative mtDNA content of INS-1 cells.

FIG. 2 illustrates the effect of exposure to a representative antiviral compound for 0-40 days on the mtDNA content of INS-1 cells.

FIG. 3 illustrates the effect of exposure to a representative antiviral compound for 40 days on basal and glucose-stimulated insulin secretion by INS-1 cells.

### DETAILED DESCRIPTION OF THE INVENTION

The present invention provides improved methods and compositions for depleting mitochondrial DNA (mtDNA) from cells, such as insulin-secreting cells and cells that are derived from pancreatic beta cells, to generate p⁰ cells and mtDNA depleted cells that are useful in the production of cybrid cells and animals. Mitochondrial DNA is depleted from insulin-secreting cells by contacting such cells with an antiviral compound. Depletion of mtDNA with antiviral compounds provides a rapid method for producing insulin secreting mitochondrial cybrid cell lines, which may be of further use in providing disease models for mitochondria associated diseases. For example, cybrid cell models of diabetes mellitus may be produced according to the methods of the present invention. Other disease models may also be produced, depending on whether mitochondria from healthy or diseased individuals are used to repopulate cells depleted of mtDNA by treatment with an antiviral compound. The invention further provides methods for preparing cybrid animals by depleting mtDNA from embryonic cells using antiviral compounds and repopulating such cells with mitochondria from a distinct cellular source.

As noted above, the invention provides methods for generating p⁰ and mtDNA depleted cells by contacting insulin-secreting cells with an antiviral compound. Insulin-secreting cells include any cells, that naturally or as a result of genetic engineering, are capable of exporting any product of an insulin gene to the extracellular environment. Methods for determining whether a cell is an insulin-secreting cell are well known and include procedures for detecting the presence of insulin or proinsulin in the extracellular milieu of a cell. For example, a radioimmunoassay (RIA) using an antibody that specifically binds to insulin may be used to identify a cell as an insulinsecreting cell. Variations on RIA such as enzyme linked immunosorbent assays and immunoprecipitation analysis, and other assays for the presence of insulin or proinsulin in a cell conditioned medium are readily apparent to those familiar with the art, and may further include assays that measure insulin secretion by cells in the presence or absence of secretagogues such as glucose, KCl, amino acids, sulfonylureas, forskolin, glyceraldehyde, succinate or other agents that may increase or decrease insulin or proinsulin in a cell conditioned medium.

Although the cells suggested for certain embodiments herein are insulin secreting pancreatic beta cells or cell lines that maintain a normal pancreatic beta cell or insulin responsive phenotype, the present invention is not limited to the use of such cells but may also include the use of other cells or cell lines that naturally or as the result of generic engineering may secrete insulin or proinsulin, including cells that secrete insulin or proinsulin in a regulated fashion. Suitable cells are cells such as, but not limited to, βTC6-βTC7, HIT-Tl5, RINm5f, βTC-1, MIN-6 and INS-1 cells (See Gadzar et al., Proc. Nat. Acad. Sci. 77:3519 (RINm5F) 1980; Newgard et al., Ann. Rev. Biochem. 64:689, 1995; Efrat et al., Diabetes 42:901-907, 1993; Civelek et al., Biochem. J. 315:1015-1019, 1996; Asfari et al., Endocrinol. 130:167-178, 1992). Other insulin secreting cell types that are useful in the present invention include cells that are derived from pancreatic beta cells, as well as freshly isolated islets of Langerhans or islet cells in primary culture.

The use of established, culture-adapted insulin secreting cell lines is preferred for use in the methods of the invention. However, primary culture cells such as insulin secreting cells obtained by explant or biopsy from an individual known or suspected of suffering from a mitochondria associated disease or from another individual, e.g., an unaffected close blood relative of a patient suffering from a mitochondria associated disorder, may be used to generate p⁰ and mtDNA depleted cells according to the present invention. This use of genetically related cells may have certain advantages for ruling out non-mitochondrial effects as causative of particular phenotypic traits in cybrid cells produced from such p⁰ cells.

Genetically altered cells, such as transfected cell lines that are insulin secreting cells as a consequence of having undergone genetic transfection, are also within the scope of cells that may be used in the present invention. Such genetically altered cells may be differentiated or undifferentiated, and may further be cells that secrete insulin in a regulated fashion. Transfection of cells with genes encoding gene products of interest such as insulin or proinsulin, and transfection of cells with genes that include regulatory elements such as, but not limited to, specifically inducible promoters, enhancers and/or transcription factor binding sites, are well known in the art. (See, e.g., Newgard et al., J. Lab. Clin. Med. 122:356-363, 1993; Hughes et al., Proc. Nat. Acad. Sci. USA 89:688-692, 1992.)

Although insulin secreting cells themselves may be used as a preferred model system for mitochondria associated disease, it may also be preferred to propagate cells capable of secreting insulin in an undifferentiated state and to induce lineage-specific differentiation prior to screening assays or diagnostic assays. Physical, biological and/or chemical agents capable of inducing differentiation in particular undifferentiated cell lines are known in the art and may be used. In any event, it is most preferred to use recipient cells that can be induced to differentiate by the addition of particular chemical (e.g., hormones, growth factors, transcription factors, etc.) or physical (e.g., temperature, exposure to radiation such as U.V. radiation, etc.) induction signals.

The present invention also provides immortal cell lines that are undifferentiated or partially differentiated, but that are capable of being induced to differentiate, and further provides fully differentiated cell lines. These cell lines have origins in immortalized beta cells or insulin-responsive cells (for example, βTC6, HIT-Tl5, RINmSf, βTC-1 and INS-1 cells). “Immortal” cell lines refers to cell lines that may be so designated by persons of ordinary skill in the art, or that may be capable of being passaged preferably an indefinite number of times, but not less than ten times, without significant phenotypic alteration.

As noted above, the present invention provides novel compositions and methods that permit rapid generation of p⁰ and mtDNA depleted cell lines using antiviral compounds. An antiviral compound may be any composition that interferes with a viral structure or a viral function. Examples of antiviral compounds include but need not be limited to nucleoside analogs, nucleic acid constructs, peptides, proteins, protease inhibitors, small molecules, cytokines and other compounds having antiviral activity. Viral functions include but need not be limited to any viral binding, infection, replication, gene expression, genetic recombination, integration, nucleic acid synthesis or particle assembly events. Viral functions may also include endocytic, phagocytic, nucleolytic, proteolytic, lipolytic, hydrolytic, catalytic, or other regulatory events. In addition, suitable antiviral compositions include those compositions that are known in the art for their antiviral activities, for instance in treating HIV infection. Suitable nucleoside analogs include AZT (3′-azido-3′deoxythymidine), ddC (dideoxycytidine), ddA (dideoxyadenosine), ddG (dideoxyguanosine), ddT (dideoxythymidine), ddI (dideoxyinosine), dideoxydidehydrothymidine, dideoxydidehydrocytidine, acycloguanosine, ganciclovir and other nucleoside analogs known to those familiar with the art including those found in Kulikowski, Pharm. World Sci. 16:127-138, 1994; Isono, Pharmac. Ther. 52:269-286, 1991; and Isono, JL. Antibiotics 41:1711, 1988; all of which are hereby incorporated by reference in their entireties. Nucleoside analogs may interfere with viral nucleic acid synthesis and replication, for example by becoming incorporated into DNA or RNA molecules complementary to viral sequences or by other mechanisms. The structures of nucleoside analogs may be non-permissive for further extension of nucleic acid strands into which the analogs have been incorporated.

Without wishing to be bound by theory, another biological activity of antiviral compounds (including nucleoside analogs) may be their inhibition of mtDNA replication. These compounds are believed to incorporate into newly synthesized mtDNA, and may also inhibit DNA polymerase gamma, a mitochondria-specific enzyme required for mtDNA replication. Regardless of whether these or other mechanisms underlie the usefulness of antiviral compounds for the generation of p⁰ cells, the present invention provides for the generation of p⁰ cells for the production of cybrid cells from any cell line or cultured cell type.

As described herein, p⁰ cells and mtDNA depleted cells may be generated by contacting cells, such as insulin secreting cells, with an antiviral compound. Although those conditions suitable for generating such cells will be evident to those skilled in the art, for any particular combination of insulin-secreting cell and antiviral compound, preferred culture conditions may be determined using various concentrations of the antiviral compounds and exposure of cells to antiviral compound(s) over various time periods. For example, by way of illustration and not limitation, human INS-1 insulinoma cells may become p⁰ cells after exposure to 25 μm ddC for 4-8 weeks in culture media supplemented with pyruvate, uridine and glucose. For other cell types or cell lines, specific concentrations of antiviral compounds and duration of exposure may be optimized using routine methodologies with which those skilled in the art will be familiar in order to generate p⁰ cells or mtDNA depleted cells. Mitochondrial DNA depletion may be readily determined using slot blot analysis or other methods known to those of ordinary skill in the art. “p⁰ cells” are cells essentially completely depleted of mtDNA, and therefore have no functional mitochondrial respiration/electron transport activity. Such absence of mitochondrial respiration may be established by demonstrating a lack of oxygen consumption by intact cells in the absence of glucose, and/or by demonstrating a lack of catalytic activity of electron transport chain enzyme complexes having subunits encoded by mtDNA, using methods well known in the art. (See, e.g., Miller et al., J. Neurochem. 67:1897-1907, 1996.) That cells have become p⁰ cells may be further established by demonstrating that no mtDNA sequences are detectable within the cells. For example, using standard techniques well known to those familiar with the art, cellular mtDNA content may be measured using slot blot analysis of 1 μg total cellular DNA probed with a mtDNA-specific oligonucleotide probe radiolabeled with, e.g., ³²p to a specific activity ≧900 Ci/gm. Under these conditions p⁰ cells yield no detectable hybridizing probe signal. Alternatively, any other method known in the art for detecting the presence of mtDNA in a sample may be used which provides comparable sensitivity.

“Mitochondrial DNA depleted” cells (“mtDNA depleted cells”) are cells substantially but not completely depleted of functional mitochondria and/or mitochondrial DNA, by any method useful for this purpose. MtDNA depleted cells are preferably at least 80% depleted of mtDNA as measured using the slot blot assay described above for the determination of the presence of p⁰ cells, and more preferably at least 90% depleted of mtDNA. Most preferably, mtDNA depleted cells are depleted of >95% of their mtDNA.

Mitochondria to be transferred to construct model systems in accordance with the present invention may be isolated from virtually any tissue or cell source. Cell cultures of all types may potentially be used, as may cells from any tissue. However, fibroblasts, brain tissue, myoblasts and platelets are preferred sources of donor mitochondria. Platelets are the most preferred, in part because of their ready abundance, and their lack of nuclear DNA. This preference is not meant to constitute a limitation on the range of cell types that may be used as donor sources.

In the examples below, platelets have been isolated by an adaptation of the method of Chomyn (Am. J. Hum. Genet. 54:966-974, 1994). However, it is not necessary that this particular method be used. Other methods are easily substituted. For example, if nucleated cells are used, cell enucleation and isolation of mitochondria isolation can be performed as described by Chromyn et al., Mol. Cell. Biol. 11:2236-2244, 1991. Human tissue from an individual with a disorder known to be associated with a mitochondrial defect that segregates with late onset diabetes mellitus may be the source of donor mitochondrial DNA.

After preparation of mitochondria by isolation of platelets or enucleation of donor cells, the mitochondria may be transplanted into p0 cells or mtDNA depleted cells using any known technique for introducing an organelle into a recipient cell, including but not limited to polyethylene glycol (PEG) mediated cell membrane fusion, cell membrane permeabilization, cell-cytoplast fusion, virus mediated membrane fusion, liposome mediated fusion, particle mediated cellular uptake, microinjection or other methods known in the art. For example by way of illustration and not limitation, mitochondria donor cells (≈1×10⁷) are suspended in calcium-free Dulbecco's modified Eagle (DME) medium and mixed with p⁰ cells (≈0.5×10⁶) in a total volume of 2 ml for 5 minutes at room temperature. The cell mixture is pelleted by centrifugation and resuspended in 150 μl PEG (PEG 1000, J. T. Baker, Inc., 50% w/v in DME). After 1.5 minutes, the cell suspension is diluted with normal p⁰ cell medium containing pyruvate, uridine and glucose, and maintained in tissue culture plates. Medium is replenished daily, and after one week medium lacking pyruvate and uridine is used to inhibit growth of unfused p⁰ cells. These or other methods known in the art may be employed to produce cytoplasmic hybrid, or “cybrid”, cell lines.

The present invention also provides insulin-responsive and insulin-secreting cybrid cell lines. In one embodiment of the invention, p⁰ cells generated from any insulin-secreting cell according to the method of the invention may be used to construct cybrid cells using mitochondria derived from a diabetic human or animal, for example a NIDDM patient or other donor exhibiting impaired insulin secretion. Such cybrid cells may be used to screen for drug candidates able to reverse or minimize defects responsible for impaired insulin secretion in NIDDM.

Another embodiment of the invention provides p⁰ cells generated using the compositions and methods of the invention for construction of xenogeneic cybrid cells. As a non-limiting example, cybrid cells may comprise human host cells and mitochondria from an animal model system. As a further non-limiting example, donor mitochondria may be provided by platelets of the BHE/cdb rat, which expresses a mutation in the mitochondrial DNA-encoded ATP synthase 6 gene, and which develops a NIDDM-like syndrome (Kim et al., 1998 Int. J Diabetes 6:1-11; Berdanier et al., 1997 Int. J Diabetes 5:27-37; Berndanier, FASEB J. 5:2139-2144, 1991).

In a preferred embodiment, the present invention provides the ability to model the precise genetic and biochemical defects in the NIDDM pancreas by providing insulin-secreting cell lines deficient in mitochondrial DNA. More particularly, the present invention provides an in vitro NIDDM model wherein depletion of mitochondrial DNA is associated with loss of glucose-stimulated insulin secretion. Cybrids may be constructed by repopulation of such mitochondrially depleted (p⁰) cells with mitochondria from normal or diseased (i.e., NIDDM) individuals. These cybrids may then be tested for restoration of glucose-stimulated insulin secretion. In a further embodiment, these cybrid cells produced from p⁰ cells generated according to the present invention may be screened for specific mitochondrial DNA mutations that may cause NIDDM. In another embodiment, these cybrid cells produced using mitochondria from NIDDM patients and exhibiting impaired insulin secretion may be used to screen for drug candidates that restore normal glucose-stimulated insulin secretion. In yet another embodiment, such cybrid cells may be used to screen for drug candidates that specifically reverse or minimize other biochemical and bioenergetic deficiencies that result from defects in NIDDM donor mitochondria.

In still another embodiment, the rapid generation of p⁰ cells that is made possible using the compositions and methods of the present invention permits construction of short-term cybrid cells, for example cybrid cells having mitochondria from NIDDM donors. Such short-term cybrids may not need to undergo transcription of mitochondrial DNA or mitochondrial replication to be useful. Instead, these cybrids can be promptly assayed for their glucose-stimulated insulin secretory responses or other phenotypic changes that may result from repopulation with potentially defective donor mitochondria.

Short-term cybrids as described above may be constructed in this manner using human P⁰ cells. Alternatively, xenogeneic cybrid cells may be produced using animal (e.g., rat) p⁰ cells and human donor mitochondria. Where stable xenogeneic NIDDM cybrid cell lines are desired, p⁰ insulin secreting cells may be transfected with suitable genes for transcription and replication of donor mitochondrial DNA. It is known that a species-specific mitochondrial transcription factor and mitochondrial DNA polymerase γ are required for transcription and replication of mitochondrial DNA, respectively (Clayton, Trends in Bioch. Sci. 16:107-111; Clayton, Int. Rev. Cytol. 141:217-232, 1992). It is further within the knowledge of one skilled in the art to stably transfect genes encoding mitochondrial transcription factor and DNA polymerase (into a cell that may be used to generate p⁰ cells for production of cybrid cell lines. For example, transformation of INS-1 insulinoma cells with donor-species genes encoding one or both of these factors may permit transcription of the donor mitochondrial genome.

In another embodiment, the invention provides a method for preparing a cybrid animal from p⁰ or mtDNA depleted embryonic cells generated using an antiviral compound according to the instant disclosure. For example by way of illustration and not limitation, mtDNA or mitochondria from a distinct biological source, such as a subject suspected of carrying a mitochondria associated disease, may be introduced into animals, creating a mosaic cybrid animal. As a further non-limiting example, a freshly fertilized mouse embryo, at about the 2 to 16 cell stage, may be washed by saline lavage from the fallopian tubes of a pregnant mouse. Under a dissection microscope, the individual cells may be teased apart, and treated with an antiviral compound, which may include a nucleoside analog, to induce a p⁰ state. Determining the appropriate duration and concentration for treatment with an antiviral compound may require the sacrifice of several embryos for Southern analysis to assure that mitochondrial function has been lost. Then, cells so treated may be repopulated with exogenous mitochondria isolated from a distinct biological source. One or more of the resulting cybrid cells may then be implanted into the uterus of a pseudopregnant female by microinjection into the fallopian tubes. At the end of gestation, the structure and/or activity of a mitochondrial gene in blood cells from one or more of the progeny may be tested to confirm that some of the mitochondria are derived from the donor. The presence of the donor mitochondrial DNA may also be confirmed by DNA sequence analysis.

Model systems made and used according to the present invention may be equally useful irrespective of whether the disease of interest is known to be caused by mitochondrial defects. Where mitochondrial disorders are a symptom of the disease, are associated with a predisposition to the disease, or have an unknown relationship to the disease, the present invention permits development of biological model systems that may be useful for screening assays to identify therapeutics or for diagnostic assays. In addition, the uses of model systems according to the present invention to determine whether a disease has an associated mitochondrial defect are within the scope of the present invention.

As a non-limiting example, the invention provides a method of detecting a disease associated with altered mitochondrial function by determining altered levels of insulin secretion by a cybrid cell line produced according to the methods disclosed herein, where such a cybrid cell line may contain mitochondria from a donor subject suspected of having a disease associated with altered mitochondrial function. Altered levels of insulin secretion, such as quantitative and/or qualitative (e.g., processing, posttranslational modification, cofactor requirements, etc.) differences in insulin secretion that may correlate with the introduction into these cells of mitochondria exhibiting altered function, may provide useful diagnostic information. Evaluation of potential mitochondria associated disease may further encompass quantitative and/or qualitative comparison of insulin secretion by a cybrid cell line that contains mitochondria from a donor subject suspected of having a disease associated with altered mitochondrial function, with insulin secretion by cybrid cells having normal mitochondria. These and similar uses of model systems according to the invention for the detection of diseases associated with altered mitochondrial function will be appreciated by those familiar with the art and are within the scope and spirit of the invention.

Model systems made and used according to the present invention may also be useful in the evaluation of antiviral compounds for their potential effects on mitochondrial function, which may further include the effect an antiviral compound may have on insulin secretion by a cell. For example by way of illustration and not limitation, the determination that an antiviral compound alters insulin secretion by an insulin secreting cybrid cell produced according to methods disclosed herein may be useful in the selection of antiviral compounds for therapeutic use in diseases, including but not limited to mitochondria associated diseases, in which altered mitochondrial function may be present as a result of the disease and/or as a consequence of any agent administered in the course of therapeutic treatment of the disease. As another example, evaluating the effect of a candidate therapeutic agent on insulin secretion by a cybrid cell produced according to the methods of the present invention may provide a method for selecting appropriate therapeutic agents for use in a subject having a disease associated with altered mitochondrial function, such as NIDDM. Accordingly, candidate therapeutic agents may be selected for their ability directly or indirectly to potentiate or impair insulin secretion.

As a further non-limiting example, model systems made and used according to the present invention may be useful for identifying agents that partially or completely restore insulin secretion to a cell exposed to an antiviral compound that inhibits insulin secretion. According to this example, impaired insulin secretion may be detected in an insulin secreting cybrid cell line produced as disclosed herein, and such an insulin secretion impaired cybrid cell line may be used to screen candidate agents by identifying those agents capable of effecting an increase in insulin secretion relative to the insulin secretion impaired state. In addition, the present invention provides model systems for selecting therapeutic agents that may be suitable for the treatment of diseases associated with altered mitochondrial function. These and similar uses of model systems according to the invention for the screening and identification of agents that counteract the effects an antiviral compound may exert on mitochondrial function, including insulin secretion, will be appreciated by those familiar with the art and are within the scope and spirit of the invention.

In addition, although the present invention is directed primarily towards model systems for diseases in which the mitochondria have metabolic defects, it is not so limited. Conceivably there are disorders wherein mitochondria contain structural or morphological defects or anomalies, and the model systems of the present invention are of value, for example, to find drugs that can address that particular aspect of the disease.

In addition, there are certain individuals that have or are suspected of having extraordinarily effective or efficient mitochondrial function, and the model systems of the present invention may be of value in studying such mitochondria. In addition, it may be desirable to put known normal mitochondria into cell lines having disease characteristics, in order to rule out the possibility that mitochondrial defects contribute to pathogenesis. All of these and similar uses are within the scope of the present invention, and the use of the phrase “mitochondrial defect” herein should not be construed to exclude such embodiments.

It is important to an understanding of the present invention to note that all technical and scientific terms used herein, unless otherwise defined, are intended to have the same meaning as commonly understood by one of ordinary skill in the art. The techniques employed herein are also those that are known to one of ordinary skill in the art, unless stated otherwise. Throughout this application various publications are referenced within parentheses. The disclosures of these publications in their entireties are hereby incorporated by reference in this application.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

The following examples are offered by way of illustration and not limitation, and are not intended to limit the scope and spirit of the invention that shall be apparent to those having skill in the art.

### EXAMPLES

### Example 1

### Treatment Of Cells With Nucleoside Analogs To Deplete Mitochondrial Dna

Cell Culture and Generation of p⁰ Cells

INS-1 rat insulinoma cells were provided by Prof. Claes Wollheim, University Medical Centre, Geneva, Switzerland, and cultured at 37° C. in a humidified 5% CO₂ environment in RPMI cell culture media (Gibco BRL, Gaithersburg, MD) supplemented with 10% fetal bovine serum (Irvine Scientific), 2 mM L-glutamine, 100 U/ml penicillin, 100 μg/ml streptomycin, 10 mM HEPES, 1 mM sodium pyruvate and 50 μM β-mercaptoethanol.

INS-1 cells were cultured for 3-60 days under conditions as described above except media were additionally supplemented with 50 μg/ml uridine and nucleoside analogs 2′3′-dideoxycytidine [ddC], 2′3′-dideoxyinosine [ddl] or 2′3′-didehydro-3-deoxythymidine [d4T] (all from Sigma) at varying concentrations (1-500 μM) diluted from 100X stock in PBS or a comparable dilution of PBS without. Media were replenished every two days. Cells were harvested at periodic intervals and assayed for insulin secretion and mtDNA content.

### Example 2

### Depletion Of Mitochondrial Dna In Cells Treated With Nucleoside Analogs

Quantification of Mitochondrial DNA by Slot Blotting

INS-1 cells, or p⁰ INS-1 cells generated using ddC as described above, were seeded into 12-well plates containing RPMI media supplemented as described above at 0.4×10⁶ cells/well and cultured at 37° C., 5% CO₂ for 2 days. Cells (0.7×106 cells/well) were rinsed with PBS and total cellular DNA was extracted using DNAzol (Molecular Research Center, Inc., Cincinnati, Ohio) according to the manufacturer's instructions. One hundred ng DNA from each cell preparation was slot-blotted onto a Zeta-Probe membrane (Bio-Rad, Hercules, Calif.) and crosslinked at 125 joules using a BioRad GS GeneLinker irradiation/energy source.

The membranes were rinsed in hybridization buffer (5X SSC, 0.1% N-laurylsarcosine, 0.02% SDS, 1% blocking solution (Boehringer Mannheim, Indianapolis)) and hybridized overnight in the same buffer at 42° C. with a [³²P]-labeled oligonucleotide probe specific for a mitochondrially encoded cytochrome c oxidase subunit I (COX-I) gene sequence and containing nucleotides 5342-6549 of the rat mitochondrial genome sequence (GenBank Accession Number X14848, Anderson et al., Nature 290:457 (1981)). This probe was radiolabeled using a Prime-a-Gene random priming kit (Promega, Madison, Wis.) according to the manufacturer's recommendations. Following hybridization, membranes were washed twice with 2X SSC/0.1% SDS and twice with 0.1X SSC/0.1% SDS and exposed to X-ray film. Mitochondrial DNA was quantified by densitometric scanning of the resulting autoradiographs.

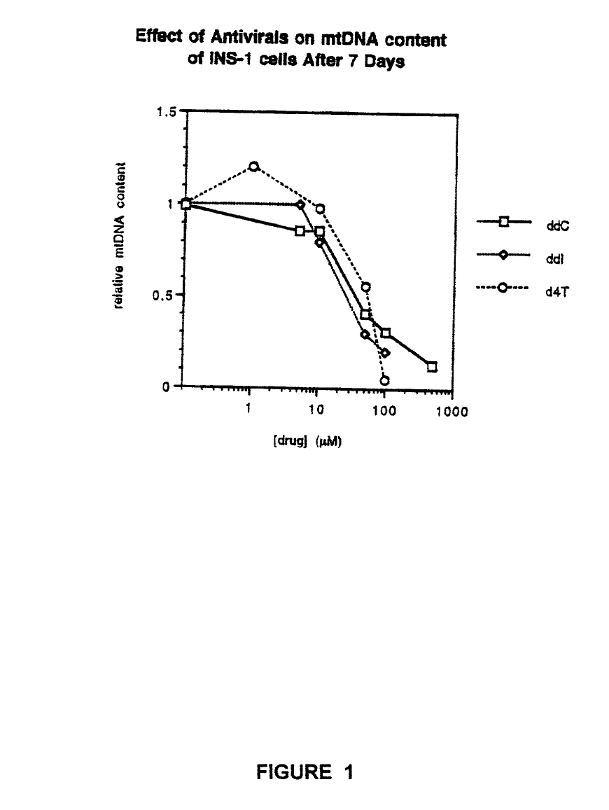

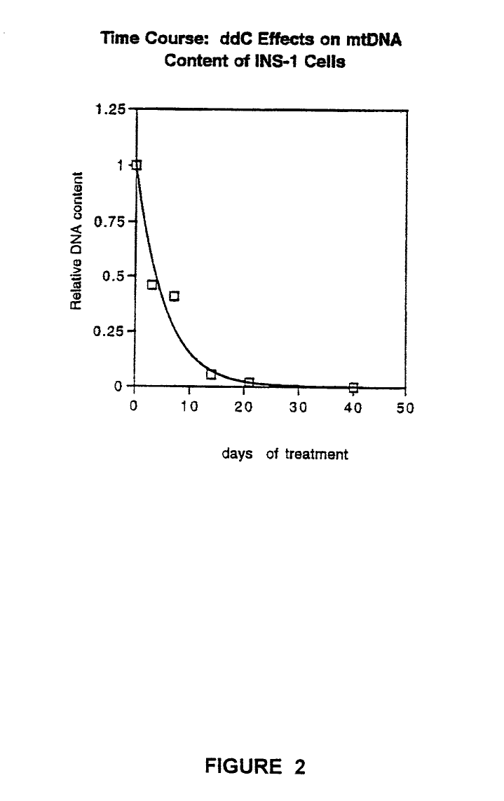

Incubation of INS-1 cells with ddC, ddI or d4T for seven days decreased mtDNA content in a dose-dependent fashion, as shown in FIG. 1. The relative mtDNA content (mean COX-I hybridization signal+SEM) of the cells, normalized to total cellular DNA, is plotted as a function of nucleoside analog concentration. The IC₅₀ for ddC was approximately 50 μM. In INS-1 cells incubated with 25 μM ddC for up to 40 days, the decline in mtDNA content was time-dependent, with a t_{1/2} of approximately three days; mtDNA was undetectable in these cells after 21 days. (FIG. 2.)

### Example 3

### Insulin Secretion By Mitochondrial Dna-Depleted Cells Generated Using Nucleoside Analogs

Insulin Secretion

INS-1 cells, or p⁰ INS-1 cells generated using ddC as described above, were seeded into 12-well plates containing RPMI media supplemented as described at 0.5×10⁶ cells/well and cultured at 37° C., 5% CO₂ for 2 days. Cells (0.7×10⁶ cells/well were rinsed with glucose-free KRH buffer (134 mM NaCl, 4.7 mM KCl, 1.2 mM KH₂PO4, 1.2 mM MgSO₄, 1.0 mM CaCl₂, 10 mM HEPES, 10 mM NaHCO₃, 0.5% BSA), then incubated in the same buffer for 1 hr at 37° C. in a humidified 5% CO₂/95% air atmosphere. Fresh KRH buffer containing 0.5 mM isobutylmethyl xanthine and the following secretagogues was added: 5 mM glucose, 10 mM glucose, 20 mM glucose, 5 mM KCl or 20 mM KCI. After an additional 1 hr at 37° C., 5% CO₂ the culture supernatants were collected. Insulin concentrations in the supernatants were measured and normalized to cell number using an insulin-specific radioimmunoassay kit (ICN Biochemicals, Irvine, Calif.) according to the manufacturer's instructions.

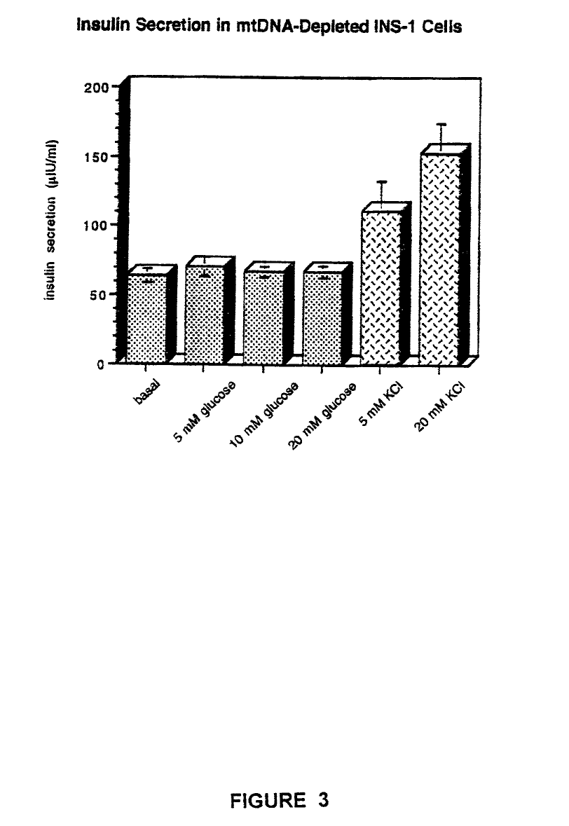

INS-1 cells normally exhibit half-maximal glucose-mediated insulin secretion at 5 mM glucose. Following treatment with ddC (10 μM for 40 days, at which time mtDNA was undetectable, no glucose stimulated insulin secretion was observed at any glucose level tested (FIG. 3). In contrast, KCl-mediated insulin secretion, which bypasses the mitochondrial component of the insulin secretory pathway, remained intact.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method of generating a p⁰ cell comprising:
contacting an insulin secreting cell with an antiviral compound.

2. A method of generating a mitochondrial DNA depleted cell comprising:
contacting an insulin secreting cell with an antiviral compound.

3. The method of either claim 1 or claim 2 wherein the antiviral compound is a nucleoside analog.

4. The method of claim 3 wherein the antiviral compound is selected from the group consisting of 2′3′-dideoxycytidine, 3′-azido-3′deoxythymidine, dideoxyadenosine, dideoxyguanosine, dideoxythymidine, 2′3′-dideoxyinosine, 2′3′didehydro-3′-deoxythymidine, dideoxydidehydrothymidine, dideoxydidehydrocytidine, ganciclovir and acycloguanosine.

5. The method of either claim 1 or claim 2 wherein the insulin secreting cell is an immortalized cell line.

6. The method of either claim 1 or claim 2 wherein the insulin secreting cell is capable of being induced to differentiate.

7. The method of either claim 1 or claim 2 wherein the insulin secreting cell is undifferentiated.

8. A method of producing a cybrid cell line, comprising the steps of:
treating an insulin secreting cell line with an antiviral compound to convert said cell line into a p⁰ cell line; and
repopulating said p⁰ cell line with isolated mitochondria to form said cybrid cell line.

9. The method of claim 8 wherein the cybrid cell line has extramitochondrial genomic DNA and mitochondrial DNA of differing biological origins.

10. The method of claim 9 wherein the cybrid cell line has extramitochondrial genomic DNA and mitochondrial DNA from xenogeneic species.

11. The method of claim 10 wherein the cybrid cell line has mitochondrial DNA from a rodent species.

12. The method of claim 11 wherein the cybrid cell line has mitochondrial DNA from a species selected from the group consisting of mouse, rat, rabbit, hamster, guinea pig and gerbil.

13. The method of claim 12 wherein the cybrid cell line has mitochondrial DNA from a BHE/cdb rat.

14. A method of producing a cybrid cell line, comprising the steps of:
treating an insulin secreting cell line with an antiviral compound to convert said cell line into a mitochondrial DNA depleted cell line; and
repopulating said mitochondrial DNA depleted cell line with isolated mitochondria to form said cybrid cell line.

15. The method of claim 14 wherein the cybrid cell line has extramitochondrial genomic DNA and mitochondrial DNA of differing biological origins.

16. The method of claim 15 wherein the cybrid cell line has extramitochondrial genomic DNA and mitochondrial DNA from xenogeneic species.

17. The method of claim 16 wherein the cybrid cell line has mitochondrial DNA from a rodent species.

18. The method of claim 17 wherein the cybrid cell line has mitochondrial DNA from a species selected from the group consisting of mouse, rat, rabbit, hamster, guinea pig and gerbil.

19. The method of claim 18 wherein the cybrid cell line has mitochondrial DNA from a BHE/cdb rat.

20. The method of either claim 8 or claim 14 wherein the antiviral compound is a nucleoside analog.

21. The method of claim 20 wherein the antiviral compound is selected from the group consisting of 2′3′-dideoxycytidine, 3′-azido-3′deoxythyrnidine, dideoxyadenosine, dideoxyguanosine, dideoxythymidine, 2′3′-dideoxyinosine, 240 3′didehydro-3′-deoxythymidine, dideoxydidehydrothymidine, dideoxydidehydrocytidine, ganciclovir and acycloguanosine.

22. The method of either claim 8 or claim 14 wherein the insulin secreting cell line to be treated with an antiviral compound is an immortalized cell line.

23. The method of any one of claims 8-19, wherein the cybrid cell line is capable of secreting insulin.

24. The method of any one of claims 8-19 wherein the cybrid cell line is capable of responding to insulin.

25. The method of any one of claims 8-19 wherein the cell line is derived from a pancreatic beta cell.

26. The method of any one of claims 8-19 wherein said cell line is an undifferentiated cell line that is capable of being induced to differentiate.

27. The method of any one of claims 8-19 wherein said isolated mitochondria are obtained from a subject known to be afflicted with a disorder associated with a mitochondrial defect.

28. The method of any one of claims 9-13 or 15-19, wherein said extramitochondrial genomic DNA has its origin in an immortal cell line, and said mitochondrial DNA has its origin in a human tissue sample.

29. The method of claim 28 wherein said human tissue sample is derived from a patient having a disease that is associated with a mitochondrial defect.

30. A method of constructing an immortal cybrid cell line, comprising the steps of:
a) treating an immortal insulin secreting cell line with an antiviral compound to convert said cell line into an immortal p⁰ cell line; and
b) repopulating said immortal p⁰ cell line with mitochondria isolated from tissue of a patient afflicted with a disorder selected from the group consisting of diabetes mellitus, Alzheimer's Disease, Parkinson's Disease, Huntington's disease, dystonia, Leber's hereditary optic neuropathy, schizophrenia, myoclonic-epilepsy-lactic-acidosis -and-stroke (MELAS), and myoclonic-epilepsy-ragged-red-fiber—syndrome (MERRF), to form said cybrid cell line.

31. A method of constructing an immortal cybrid cell line, comprising the steps of:
a) treating an immortal insulin secreting cell line with an antiviral compound to convert said cell line into an immortal mitochondrial DNA depleted cell line; and
b) repopulating said immortal mitochondrial DNA depleted cell line with mitochondria isolated from tissue of a patient afflicted with a disorder selected from the group consisting of diabetes mellitus, Alzheimer's Disease, Parkinson's Disease, Huntington's disease, dystonia, Leber's hereditary optic neuropathy, schizophrenia, myoclonic-epilepsy-lactic-acidosis-and-stroke (MELAS), and myoclonic-epilepsy-ragged-red-fiber syndrome (MERRF), to form said cybrid cell line.

32. A method of preparing a cybrid animal, comprising the steps of:
a) treating embryonic cells isolated from a multicellular, non-human animal with an antiviral compound, thus converting said cells to a p⁰ state; and
b) repopulating said p⁰ embryonic cells with mitochondria isolated from another cell source, to produce said cybrid animal.

33. A method of preparing a cybrid animal, comprising the steps of:
a) treating embryonic cells isolated from a multicellular, non-human animal with an antiviral compound, thus converting said cells to a mitochondrial DNA depleted state; and
b) repopulating said mitochondrial DNA depleted embryonic cells with mitochondria isolated from another cell source, to produce said cybrid animal.

34. A method of detecting a disease associated with altered mitochondrial function comprising:
treating an insulin secreting cell line with an antiviral compound to convert said cell line into a mitochondrial DNA depleted cell line or a p⁰ cell line;
repopulating said mitochondrial DNA depleted cell line or p⁰ cell line with mitochondria from a donor subject suspected of having a disease associated with altered mitochondrial function to produce a cybrid cell line;
determining altered levels of insulin secretion by said cybrid cell line; and
therefrom identifying the mitochondria donor subject as having a disease associated with altered mitochondrial function.

35. A method of detecting a disease associated with altered mitochondrial function comprising:
treating an insulin secreting cell line with an antiviral compound to convert said cell line into a mitochondrial DNA depleted cell line or a p⁰ cell line;
repopulating said mitochondrial DNA depleted cell line or p⁰ cell line with mitochondria from a donor subject suspected of having a disease associated with altered mitochondrial function to produce a cybrid cell line;
comparing altered levels of insulin secretion by said cybrid cell line to insulin secretion by an insulin secreting cell line having mitochondria from a subject with normal mitochondrial function; and
therefrom identifying the mitochondria donor subject as having a disease associated with altered mitochondrial function.

36. A method of evaluating an antiviral compound for its effect on mitochondrial function, comprising:
treating an insulin secreting cell line with an antiviral compound to convert said insulin secreting cell line into a mitochondrial DNA depleted cell line or a p⁰ cell line;
repopulating said mitochondrial DNA depleted cell line or p⁰ cell line with mitochondria to produce a cybrid cell line; and
determining insulin secretion by said cybrid cell line in the presence or absence of an antiviral compound, therefrom identifying an effect of said antiviral compound on mitochondrial function.

37. The method of claim 36 wherein said mitochondria are from a subject suspected of having a disease associated with altered mitochondrial function.

38. The method of claim 36 wherein said cybrid cell line has extramitochondrial genomic DNA and mitochondrial DNA of differing biological origins.

39. The method of claim 36 wherein said cybrid cell line has extramitochondrial genomic DNA and mitochondrial DNA from xenogeneic species.

40. The method of claim 36 wherein said cybrid cell line has mitochondrial DNA from a rodent species.

41. The method of claim 36 wherein said cybrid cell line has mitochondrial DNA from a species selected from the group consisting of mouse, rat, rabbit, hamster, guinea pig and gerbil.

42. The method of claim 36 wherein said cybrid cell line has mitochondrial DNA from a BHE/cdb rat.

43. A method of identifying an agent that at least partially restores insulin secretion to a cell exposed to an antiviral compound which inhibits insulin secretion, comprising:
treating an insulin secreting cell line with an antiviral compound to convert said cell line into a mitochondrial DNA depleted cell line or a P⁰ cell line;
repopulating said mitochondrial DNA depleted cell line or p⁰ cell line with mitochondria to produce a cybrid cell line;
contacting said cybrid cell line with a candidate agent capable of at least partially restoring insulin secretion to said cybrid cell line;
detecting an increase in insulin secretion by said cybrid cell line; and
therefrom identifying an agent that partially restores insulin secretion.

44. A method for selecting a therapeutic agent suitable for use in a subject having a disease associated with altered mitochondrial function, comprising:
treating an insulin secreting cell line with an antiviral compound to convert said cell line into a mitochondrial DNA depleted cell line or a p⁰ cell line;
repopulating said mitochondrial DNA depleted cell line or p⁰ cell line with mitochondria from a subject having a disease associated with altered mitochondrial function to produce a cybrid cell line;
detecting the level of insulin secretion by said cybrid cell line;
contacting said cybrid cell line with a candidate therapeutic agent;
detecting the effect of said candidate therapeutic agent on insulin secretion by said cybrid cell line; and
therefrom determining the suitability of the therapeutic agent.

45. A method for selecting a suitable therapeutic agent for use in a subject having a disease associated with impaired insulin secretion, comprising:
treating an insulin secreting cell line with an antiviral compound to convert said cell line into a mitochondrial DNA depleted cell line or a p⁰ cell line;
repopulating said mitochondrial DNA depleted cell line or p⁰ cell line with mitochondria from a subject having a disease associated with impaired insulin secretion to produce a cybrid cell line;
detecting the level of insulin secretion by said cybrid cell line;
contacting said cybrid cell line with a candidate therapeutic agent;
detecting the effect of said candidate therapeutic agent on insulin secretion by said cybrid cell line; and
therefrom determining the suitability of the therapeutic agent.
